(19) United States
(12) Patent Application Publication
(10) Pub. No.: US 2010/0249577 A1
(43) Pub. Date: Sep. 30, 2010

(54) Synergistic Electromagnetic Tracking With TMS Systems

(76) Inventor: Mark R. Schneider, Williston, VT (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/612,130

(22) Filed: Nov. 4, 2009

Related U.S. Application Data
(60) Provisional application No. 61/162,895, filed on Mar. 24, 2009.

(51) Int. Cl. A61B 5/05

(52) U.S. Cl. 600/424

(57) ABSTRACT

A system for tracking the location of a magnetic stimulation coil.

### CROSS-REFERENCE TO RELATED APPLICATIONS

This application claims the benefit of U.S. Provisional Application Ser. No. 61/162,895, filed Mar. 24, 2009, which is fully incorporated by reference herein.

### FIELD OF THE INVENTION

The present invention relates to the melding of magnetic tracking into transcranial magnetic stimulation (TMS) designs. It employs the magnetic components of the TMS system to perform tracking with a minimal amount of change. It thus provides a lower cost, more accurate tracking methodology then presently provided using add on optical or magnetic tracking devices.

### BACKGROUND OF THE INVENTION

Transcranial magnetic stimulation (TMS) is an FDA approved procedure that uses magnetic fields to stimulate nerve cells in the brain in the hope of improving chronic depression symptoms. TMS is a noninvasive method to excite neurons in the brain. There are different ways to perform TMS, but in general, a large electromagnetic coil is placed against your scalp near your forehead. The electromagnet induces weak electric currents in the brain tissue via rapidly changing magnetic fields (electromagnetic induction), thus creating painless electric currents that stimulate nerve cells in the region of the brain involved in mood regulation and depression. This way, brain activity can be triggered with minimal discomfort, and the functionality of the circuitry and connectivity of the brain can be studied. A variation of TMS, known as repetitive transcranial magnetic stimulation (rTMS) can produce longer lasting changes. (rTMS therapy for drug-resistant depression is approved by Health Canada).

Depression is usually a very treatable condition. Often, standard treatment with antidepressant medications, psychotherapy or electro convulsive therapy can help improve even severe cases of depression. But when standard methods fail, TMS is the least invasive of the brain stimulation procedures recently approved by the Food and Drug Administration as a depression treatment. It requires no surgery or implantation of electrodes or a nerve stimulator.

A large number of studies using TMS and rTMSA have been conducted for a variety of other neurological and psychiatric conditions, besides depression, including:
Stroke
Nonfluent aphasia
Tinnitus
Parkinson's Disease
Dystonia
Amyotrophic lateral sclerosis
Epilepsy
Migraine
Dysphasia
Hemispatial neglect
Phantom limb
Chronic pain
Obsessive-compulsive disorder
Auditory Hallucinations associated with Schizoaffective Disorders

In general during transcranial magnetic stimulation, an electromagnetic coil is placed against your scalp on an area near your forehead, often on the left side. To produce the stimulating pulses, the electromagnetic coil is switched off and on repeatedly, sometimes up to 10 times a second. The magnetic pulses create painless electrical currents in your brain. These currents stimulate nerve cells in the region of your brain involved in mood regulation and depression. In some types of TMS, brain activity is suppressed. In other types, brain activity is increased. In present systems, this results in a tapping or clicking sound that usually lasts for a few seconds, followed by a pause. This process is repeated for the duration of the treatment session, which lasts about 30 to 40 minutes.

The effects of TMS can be divided into two types depending on the mode of stimulation:
Single or paired pulse (TMS)—The pulse(s) causes a population of neurons in the neocortex to depolarise and discharge an action potential. If used in the primary motor cortex, it produces a motor-evoked potential (MEP) which can be recorded on electromyography (EMG). If used on the occipital cortex, optical disturbances might be detected. In most other areas of the cortex, the participant does not consciously experience any effect, but his or her behaviour may be slightly altered, or changes in brain activity may be detected. These effects do not outlast the period of stimulation. A review of TMS can be found in the Handbook of Transcranial Magnetic Stimulation. [1]
Repetitive TMS (rTMS)—produces effects which last longer than the period of stimulation. rTMS can increase or decrease the excitability of corticospinal or corticocortical pathways depending on the intensity of stimulation, coil orientation and frequency of stimulation. A recent review of rTMS can be found in Fitzgerald et al, 2006. [2]

TMS and rTMS also provide an important diagnostic technique in neuroscience for localizing brain function. Neuronal activity in a particular region can be virtually lesioned and the results of the lesioning can provide important diagnostic information. This can be done in two-ways:
Online TMS—where subjects perform the task and at a specific time point (usually in the order of 1-200 ms) of the task, a TMS pulse is given to a particular part of the brain. This should affect the performance of the task specifically, and thus demonstrate that this task involves this part of the brain at this particular time point. The advantage of this technique is that any positive result can provide a lot of information about how and when the brain processes a task, and there is no time for a placebo effect or other brain areas to compensate. The disadvantage of this technique is that in addition to the location of stimulation, one also has to know roughly when the part of the brain is responsible for the task so lack of effect is not conclusive.
Off line rTMS—where performance at a task is measured initially and then rTMS is given over a few minutes, and the performance is measured again. This technique has the advantage of not requiring knowledge of the timescale of how the brain processes. However off line repetitive TMS is very susceptible to the placebo effect.

While it's considered generally safe, it's not without some risks. Common side effects and adverse health problems associated with TMS include, but may not be limited to:
Headache
Scalp discomfort at the site of stimulation
Tingling, spasms or twitching of facial muscles
Light headedness
Discomfort from noise during treatment

Other good sources of information are:
http://www.biomag.hus.fi/index.html

Magnetic stimulation also finds uses in other parts of the body. While this disclosure is written towards the incorporation of tracking within a TMS system, tracking can also be performed with any other magnetic stimulation system, as known in the art.

### Present TMS Design Techniques

An enclosed coil of wire is held to the head. When the coil is energized by the rapid discharge of a large capacitor, a rapidly changing current flows in its windings. This produces a magnetic field oriented orthogonally to the plane of the coil. The magnetic field passes unimpeded through the skin and skull, inducing an oppositely directed current in the brain that flows tangentially with respect to the skull. The current induced in the structure of the brain activates nearby nerve cells in much the same way as currents applied directly to the cortical surface. The path of this current is complex to model because the brain is a non-uniform conductor with an irregular shape. With stereotactic MRI-based control, the precision of targeting TMS can be improved to a few millimeters [3].

A typical TMS system has the following characteristics:
magnetic field: often about 2 tesla on the coil surface and 0.5 T in the cortex
current rise time: zero to peak, often around 70-100 microseconds
waveform: monophasic or biphasic—the monophasic waveform generates field in one direction, whereas the biphasic waveform generates field in two phases, one positive and one negative (see FIGS. 1, (a), (b) and (c)).
repetition rate for rTMS: below 1 Hz (slow TMS), above 1 Hz (rapid-rate TMS)

The design of transcranial magnetic stimulation coils used in either treatment or diagnosis are numerous. The main differentiating characteristics are:
the type of material used to construct the core of the coil
the geometry of the coil configuration
the pulse shape produced by the coil.

Coil cores are typically air core or ferrite (solid) core. Ferrite core designs result in a more efficient transfer of electrical energy into the magnetic field, with a substantially reduced amount of energy dissipated as heat. Coil(s) geometry also results in variations in the focus shape, and depth of cortical penetration of the magnetic field. A number of different coil geometries exist, each of which produce different magnetic field patterns. Some examples:
round coil: the original type of TMS coil
figure-eight coil (i.e. butterfly coil): results in a more focal pattern of activation
double-cone coil: conforms to shape of head, useful for deeper stimulation
Deep TMS (or H-coil): currently being used in a clinical trial for the treatment of patients suffering from clinical depression.

Coupling this with different waveform shapes (e.g., width or duration of the magnetic field pulse) results in many variations in the biophysical characteristics of the resulting magnetic pulse. [4]

There are a number of major manufacturers of general purpose TMS and repetitive TMS equipment, including:
The Magstim Company, UK
MagVenture A/S, Denmark
Nexstim, Finland
Schwarzer, Germany
Neuronetics, Inc., USA

These devices are expensive (US $25,000-500,000, depending on capability). As of October 2008, a Neuronetics Inc. NeuroStar system has been approved for use by the FDA for use in adult patients with major depression who have previously tried medication and not improved satisfactorily. Some of these systems incorporate optical tracking capability (Magstim and Nexstim) for positioning of the coil. These systems are costly, large and provide coil tracking that is offset from the coil. Literature searches indicate that magnetic tracking is also used, but mostly in research at this point and mostly for digitizing. This patent discloses a method that integrates magnetic tracking directly into the TMS system, at little additional cost, and provides coil tracking right at the coils. Additional benefits include small size, and the ability to provide additional location measurements that are referenced to the coils. These can be used for digitizing the cranium or providing fiducials for registration purposes.

Examples of magnetic AC tracking systems with a plurality of generating and sensing elements are disclosed in U.S. Pat. No. 3,868,565 to Kuipers, U.S. Pat. No. 4,054,881 to Raab, and U.S. Pat. No. 4,737,794 to Jones. Tracking systems developed by Mednetix and assigned to Northern Digital of Canada are also known and incorporated herein. Additionally, other position and orientation systems using AC magnetic fields are disclosed in U.S. Pat. No. 6,980,921 to Anderson et al., U.S. Pat. No. 6,073,043 to Schneider et al. (the “'043 patent”), and U.S. Pat. No. 6,427,079 to Schneider et al. (the “'079” patent), all of which are incorporated herein by reference.

Another method is disclosed by U.S. Pat. No. 6,246,231 to Ashe (the “'231 patent”). This describes shielding field generators with high permeability shields in a relatively flat housing. The shield is placed behind the field generators and “reflects” the generated field (and theoretically doubles the field strength) so that no field is observed below the shield and it eliminates interference from fields and/or objects below it. This allows accurate tracking above the field generators. This is incorporated herein by reference.

Examples of pulsed-DC systems with a plurality of generating and sensing elements are disclosed in U.S. Pat. No. 4,945,305 to Blood (the “305 patent”), U.S. Pat. No. 5,453,686 to Anderson (the “686 patent”) and U.S. Pat. No. 6,754,596 to Ashe (the “'596 patent”), all of which are incorporated herein by reference.

Other electromagnetic tracking methods are disclosed in patents assigned to Calypso Medical, such as published patent application U.S. 20020193685 and 20050195084. These patents use wireless excitable magnetic markers for localization, and are incorporated herein.

In all of these tracking methods, magnetic fields are generated by forcing a current through a number of coils of wire (transmission) and measuring the induced voltage across a number of other coils of wire (sensing). Here coils can mean, in general, any conductive “loop”, be it a copper wire, a printed circuit board trace, etc. Also, however, field generation can take other forms, such as a moving magnet or electromagnet, and field reception can be done with semiconductor devices and other magnetically sensitive components.

Certain other requirements have to be met, including the product of sensing and transmitting coils must be no less than the degrees of freedom of location parameters desired to be determined, the various transmission coils cannot generate identical field geometries in the same tracking volume (no additional information gained), etc. The magnetic tracking systems can all work in either direction, i.e., multiple transmitter coils with a single coil sensor, multiple sensor coils with a single transmitter coil, multiple transmitter and sensor coils, etc. Additionally, location parameters can be determined from either the transmitter reference frame or the sensor reference frame. These restrictions are known in the art.

The disclosures of all publications mentioned in the specification are hereby incorporated in their entirety by reference.

### BRIEF DESCRIPTION OF THE DRAWINGS

The present invention will be understood and appreciated more fully from the following detailed description, taken in conjunction with the drawings and appendices in which:

FIG. 1 is a simplified illustration of a TMS system incorporating TMS coil tracking constructed and operative in accordance with a preferred embodiment of the present invention.

FIG. 2 is a simplified illustration of various tracked styli used in conjunction with the apparatus of FIG. 1.

FIG. 3 is a simplified illustration of a tracked movement measurement method used in conjunction with the apparatus of FIG. 1.

FIG. 4 is a simplified illustration of a TMS system incorporating additional coils along with the TMS coil tracking constructed and operative in accordance with another preferred embodiment of the present invention.

FIG. 5 is a simplified illustration of a TMS system incorporating additional TMS coils for both tracking and enhanced focus of the TMS field, constructed and operative in accordance with another preferred embodiment of the present invention.

FIG. 6 is a block diagram of a system for calibrating a TMS coil to improve focus accuracy in accordance with a preferred embodiment of the present invention.

FIG. 7 is a flowchart of the calibration method from FIG. 6 and an illustration of how the calibration is used in accordance with a preferred embodiment of the present invention.

### DESCRIPTION OF THE PREFERRED EMBODIMENTS

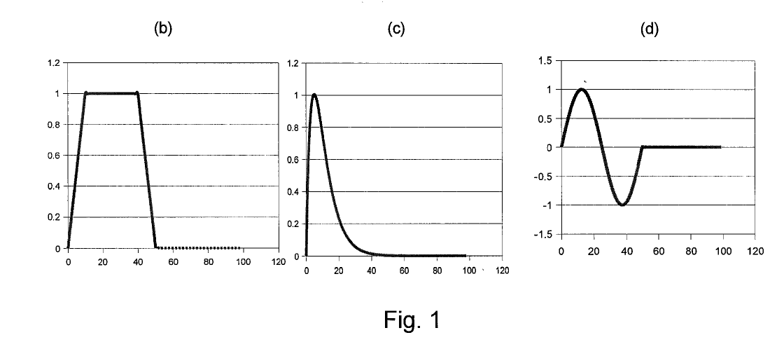

In one preferred embodiment (FIG. 1 (a)) the TMS coil 10 is the only magnetic generating element. It is typically driven by a waveform generator 11, capable of producing high voltages and currents such as to produce waveforms shown in FIGS. 1 (b), (c) and (d). The generator 11 is comprised of a source of waveforms 13 and a means of generating high currents and voltages, symbolized by 12. As is known in the art, generator 11 can also be constructed from power supplies, capacitor banks and diode waveform shapers. The generator 11, in turn, is controlled by controller 14, which sets waveform generator amplitudes, durations, and repetition rates, and provides synchronization and other capabilities to the user. User inputs 15 set the aforementioned parameters and may be manually and/or algorithmically set. Examples of TMS systems 27 are disclosed in U.S. Pat. No. 5,738,625 to Gluck, U.S. Pat. No. 4,940,453, U.S. Pat. No. 5,047,005 and U.S. Pat. No. 5,116,304 to Cadwell, U.S. Pat. Nos. 6,827,681 and 7,008,370 to Tanner el al., U.S. Pat. No. 6,572,528 to Rohan et al., and U.S. Pat. No. 7,087,008 to Fox et al. and are incorporated by reference. The waveforms 1(b), (c) and (d) have been discovered to be appropriate for performing the TMS procedure. As such, their shape and amplitude are not under the magnetic tracking systems control. Tracking using these waveforms can be performed in either a synchronized or un-synchronized manner.

Waveform repetition rate, coil excitation amplitude, frequency of the waveform and duration of the TMS treatment are all under control of the controller 14. The clinician typically sets these parameters, via user inputs 15, based on the required treatment. Amplifier 12 is typically comprised of a power supply and a capacitor bank; the capacitor bank is charged up by the power supply and switched to the TMS coil 10 via a semiconductor switch. This switching, as well as the charging of the capacitor, the duty cycle, etc are under the control of controller 14, which is typically a computer.

In the preferred synchronized embodiment the TMS system 27 is modified to provide a real time measurement of the current flowing through TMS coil 10. This is achieved by monitoring the voltage across resistor 26 using A/D 28, where the voltage is a scaled version of the current, said scaling set by the resistance of the resistor. The A/D 28 results are passed to controller 14, and this information is passed to processor 19. Other current measurement methods, such as current transformers, can be used, as is known in the art. This allows the field strength of the magnetic field generated by the TMS system 27 to be monitored and the resulting position and orientation (P&O) calculation to be corrected for variations in current, as is known in the art.

Additionally, coil 10's geometry is characterized before use, typically at the time of manufacture, using calibration techniques known in the art. These characteristics may include such simple things as coil size, inner and outer diameter, etc, to a complete mapping of the magnetic field structure produced by the coil in 3 dimensions. U.S. Pat. No. 6,427,079 to Schneider et al., U.S. Pat. No. 6,484,118 to Govari and U.S. Pat. No. 6,335,617 to Osadchy, et al. disclose these methods and are incorporated by reference.

Because the current waveform is monitored by Processor 19 via A/D 28 the sensed signals from sensor array 20 can be synchronized to the TMS excitation waveform. This makes certain signal processing algorithms performed in processor 19, for example, easier to perform or enhances their performance. These algorithms might include synchronous demodulation (coherent detection), waveform integration, FFT, etc, where knowing the start and duration and end of the waveform provides some numerical advantage.

A non-synchronized embodiment requires the constant monitoring of the data from the sensors 21 forming sensor array 20 by either signal processing block 17 or other signal processing performed in processor 19. Cross correlation with a known waveform, zero crossing and threshold detectors and other means known in the art can be utilized to detect the onset of the TMS waveform. Once onset is detected, the methods noted above can also be used. A hybrid method that uses processor 19 to monitor a level, as from a switch input associated with user inputs 15 or one generated by controller 14, could also be used to indicate waveform onset via communication over linkage 22. The current through the TMS coil 10 could be calculated using the method disclosed in U.S. Pat. No. 6,427,079 to Schneider et al. An array 20 of magnetic field sensing devices are positioned in a known manner to a known reference location to determine the location parameters of TMS coil to sensor array. Hall effect and GMR devices are possible choices for directly measuring the magnetic field. Magnetometers may also be used. Other methods are known in the art. If the generated fields look like FIG. 1, (b), (c) or (d) then the received signal will have the same shape. In the preferred embodiment, coils 21 are used to sense the field. These coils can be wire wound or formed on printed circuit boards to provide regular, repeatable sensing devices. Coil type sensors, however, measure the derivative of the field generated, so that tracker components 24 must be optimized for this form of measurement. In this case, the sensed fields will look like FIGS. 1, (e), (f) and (g), respectively, if the field generated looked like (b), (c) and (d). Of course, different variations of TMS allow for single pulse or repetitive pulsed waveforms (multiples of those shown), with different duty cycles, etc. These or any other waveforms used in the future are consistent with this disclosure.

In FIG. 1, tracker 24 consists of 9 amplifiers 25 that amplify the sensor signals, which are then processed by signal processor 17. Signal processor 17 can consist of multiplexers, filtering, integrators, differentiators, correlators, synchronous demodulators, etc or other signal processing components known in the art. From there an analog to digital converter 18 converts the signal processed signals to a digital format for use by processor 19. As is known in the art, other circuit and system topologies are possible, such as multiplexing amplified sensor signals down to one channel, or multiplexing separate processed signals down to one A/D channel, as implied by FIG. 1. Separate channels made up of sensor, amplifier, signal processor and A/D are also feasible. In the preferred embodiment, 9 identical amplifiers 25 feed 9 identical filter blocks 17 to minimize noise outside the bandwidth of interest. The filter characteristics are controlled by processor 19 which selects filter characteristics based on the wave shape of the TMS coil excitation. This information is passed to processor 19 form controller 14 via interface 22. Interface 22 can be any agreed upon interface. The preferred embodiments consist of standards like USB and Ethernet.

Processor 19 controls the collection of the sensor signals, can perform additional signal processing and can compute the location parameters of the TMS coil array 20. It can also interface 22 to controller 14 to provide synchronization and other control as is known in the art. Processor 19 can also provide user interface 23 such as accepting user commands, outputting of the location parameters, etc. In the preferred embodiment, processor 19 is a digital signal processor (DSP), although any device capable of computation, such as a microprocessor, processor core, application, specific integrated circuit (ASIC), gate array, personal computer, etc may be used. The processor 19 also contains storage for data, software algorithms, etc. The preferred user interface 23 is a monitor and keyboard, but direct connection to another computer via USB or Ethernet is possible. Processor 19 interfaces to other tracker 24 components such as A/D 18, signal processor 17 over an internal bus.

In the preferred embodiment where there is only one TMS coil 10 to be tracker, only 5 degrees of freedom (position x, y and z and orientation azimuth and elevation) can be determined for the TMS coil with respect to the sensor array. The sensor coils 21 are placed and oriented in a known configuration in the array 20. Coils could be concentric, orthogonal, or not, as is known in the art. The algorithms for calculating the location parameters can be least squares algorithms based on dipole equations, mappings of the field components of the TMS coil, or any other method known in the art, such as U.S. Pat. No. 6,980,921 to Anderson et al., U.S. Pat. No. 6,4270,79 to Schneider et al., U.S. Pat. No. 4,710,708 to Rorden et al., U.S. Pat. No. 6,484,118 to Govari and WO 01/69594 to Schneider, all incorporated by reference.

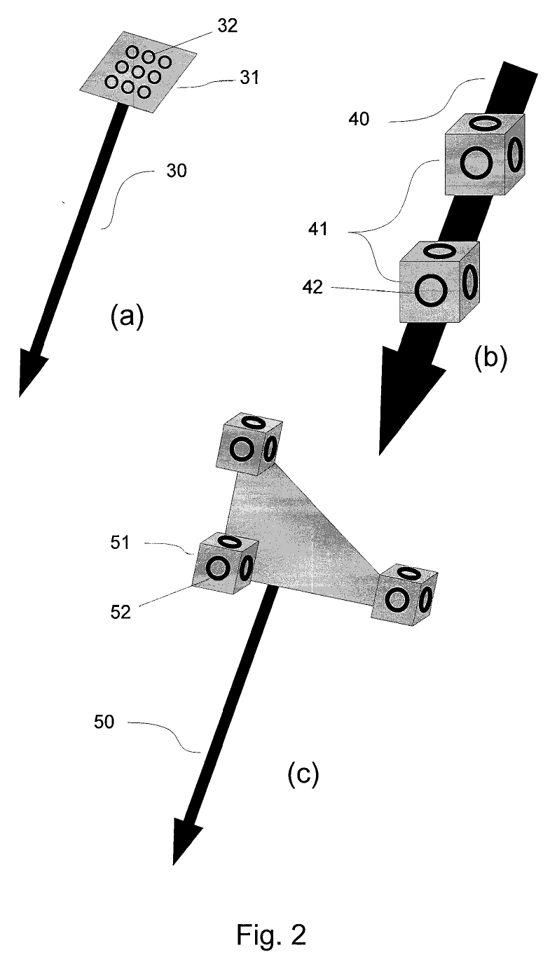

Other sensor arrays can be tracked for other purposes. One example would be a stylus for digitizing or selecting points. This could be constructed as shown in FIG. 2, (a)-(c). Styli 30, 40 or 50 have sensor arrays attached to them. In FIG. 2(a), the sensor array 31 consists of a planar set of 9 sensor coils 32, similar to array 21. In FIG. 2(b), the sensor array 41 consists of two sets of 3, roughly orthogonal sensor coils 42. These coil sets could also be concentric. In FIG. 2(c), the sensor array 51 consists of three sets of 3, roughly orthogonal sensor coils 52. These coil sets could also be concentric. Stylus calibrations and constructions are well known in the art. As noted earlier, sensors can be wire loops or semiconductor devices, as required. Calibration of the styli can be preformed using methods known in the art. For example, see http://studierstube.icg.tu-graz.ac.at/thesis/bakk_mooslechner.pdf.

As noted in Tanner et al., and Cadwell, evoked potentials can be measured using electrodes to monitor TMS therapy targeting. Along with evoked potentials, motor skills can be evaluated, which are the visible results of the evoked potentials. These are disclosed in “Repetitive transcranial magnetic stimulation-induced corticomotor excitability and associated motor skill acquisition in chronic stroke,” Kim Y H,_You S H,_Ko M H,_Park J W,_Lee K H,_Jang S H,_Yoo W K,_Hallett M.,_{—}Stroke. 2006 June; 37 (6):1471-6. Epub 2006 May 4, “Stroke Patients Benefit from Transcranial Magnetic Stimulation,” http://www.medicalnewstoday.com/articles/15992.php,

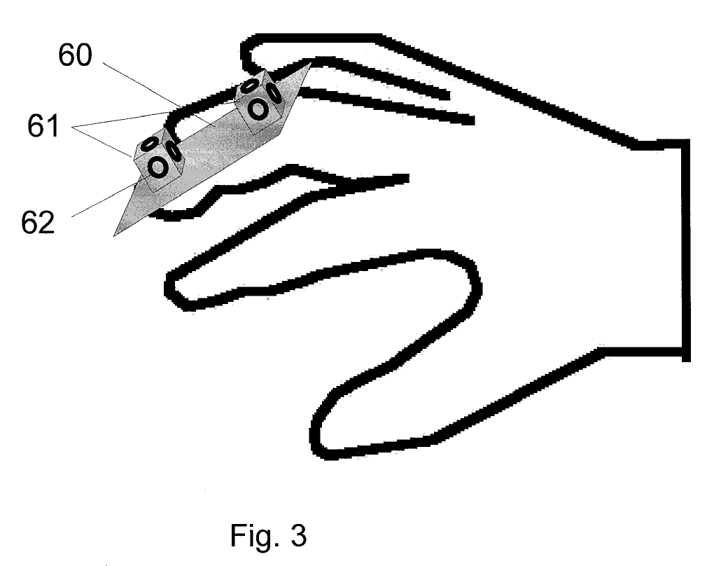

“Modulation of muscle responses evoked by transcranial magnetic stimulation during the acquisition of new fine motor skills,” A. Pascual-Leone, D. Nguyet, L. G. Cohen, J. P. Brasil-Neto, A. Cammarota and M. Hallett, Journal of Neurophysiology, Vol 74, Issue 3 1037-1045, and “Transcranial magnetic stimulation and the motor learning-associated cortical plasticity,” Milos Ljubisavljevic, Experimental Brain Research Volume 173, Number 2/August, 2006, among others. Equipment for evaluating motor skills in real time while monitoring the effects of TMS on a patient are shown in FIG. 3. In a preferred embodiment, movement monitor 60 is attached to a finger so that stimulation of the portion of the cortex responsible for finger movement could be verified. Monitor 60 consists of two sensor arrays 61, each comprised of 3, roughly orthogonal sensor coils 62.

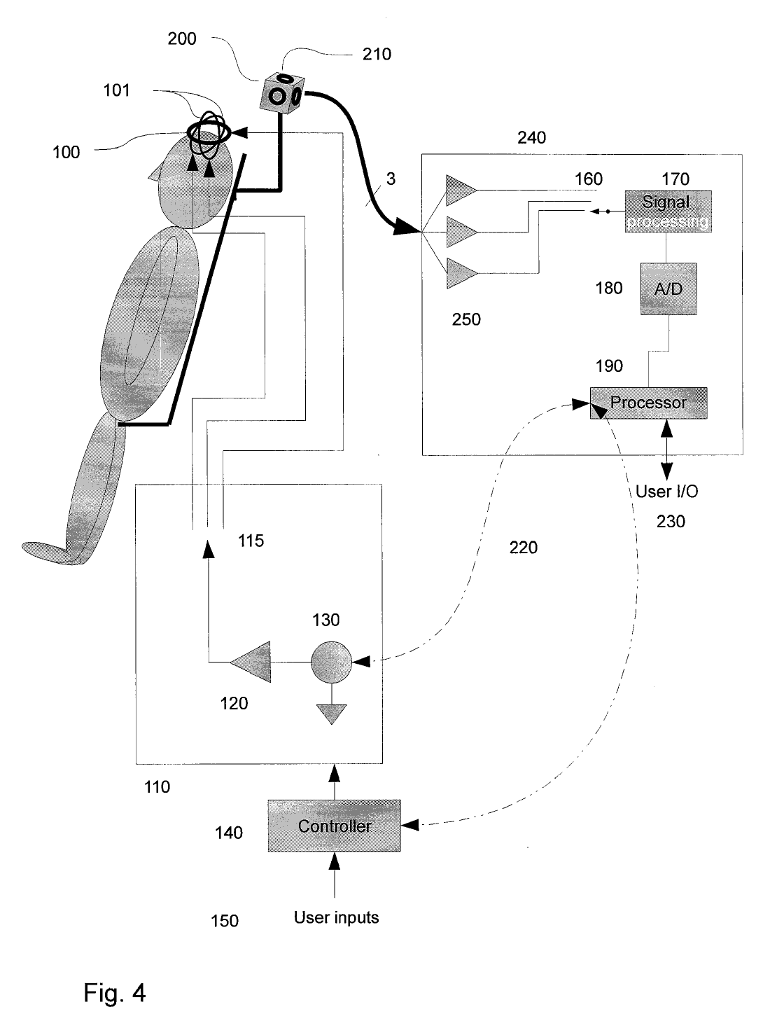

In another preferred embodiment (FIG. 4) additional field generating coils 101 and 102 are mounted adjacent to the TMS coil 100 in known locations and orientations. The generator 110 is comprised of a source of waveforms 130 and a means of generating high currents and voltages, symbolized by 120. Additionally, multiplexer 115, controlled by controller 140, selects which of the coils 100, 101, 102 receives a time multiplexed signal. It is not required that coils 101 and 102 receive the same level of excitation, or even the same waveform, as coil 100. Also, as is known in the art, coils 101 and 102 could be frequency multiplexed, with appropriate changes to the tracker 240. Coils 101 and 102 are there for tracking purposes only. The generator 130, is controlled by controller 140, which sets waveform generator amplitudes, durations, coil selection and repetition rates, and provides synchronization and other capabilities to the user. User inputs 150 set the aforementioned parameters and may be manually and/or algorithmically set. In the preferred embodiment, controller 140 sequences the coil excitations by itself, and passes the current measurement from resistor 126 to controller 140 via A/D 280. Controller 140 then communicates with processor 190 via communication link 220 to provide this information.

If the two additional transmitter coils are mounted mostly orthogonally and concentrically, then closed form dipole based algorithms can be used for determining 6DOF location parameters if the sensor array 200 is comprised of 3 orthogonal, concentric coils. Known algorithms to perform this can be found in U.S. Pat. No. 5,307,072 to Jones, Jr., among others, and is incorporated within. These coils do not have to be the same size, generate the same field or have a similar excitation as the TMS coil. Other sensor arrays consisting of a distributed set of 3 or more planar coils as in 31, or multiple sets of orthogonal, concentric coils as in 60, could also be used, but with other algorithms that have already been incorporated earlier. Once again, additional sensor arrays could be tracked for other purposes, such as digitizing or movement detection.

An array 200 of sensor coils 210 (or other devices as mentioned previously) are positioned in a known manner to a known reference location to determine the location parameters of the TMS coils 100-102 to sensor array 200. Many methods for measuring the induced voltages at the array of sensor coils 210 are known in the art and incorporated herein. In FIG. 4, tracker 240 consists of 3 amplifiers 250 that amplify the sensor signals, which are then processed by signal processor 170. Signal processor 170 can consist of multiplexers, filtering, integrators, differentiators, correlators, synchronous demodulators, etc. or other signal processing components known in the art. From there an analog to digital converter 180 converts the signal processed signals to a digital format for use by processor 190. As is known in the art, other circuit and system topologies are possible, such as multiplexing amplified sensor signals down to one channel, or multiplexing separate processed signals down to one A/D channel, as implied by FIG. 4. Separate channels made up of sensor, amplifier, signal processor and A/D are also feasible. In the preferred embodiment, 3 identical amplifiers 250 feed 3 identical filter blocks 170 to minimize noise outside the bandwidth of interest. The filter characteristics are controlled by processor 190 which selects filter characteristics based on the wave shape of the TMS coil excitations. This information is passed to processor 190 from controller 140 via interface 220. Interface 220 can be any agreed upon interface. The preferred embodiments consist of standards like USB and Ethernet.

Processor 190 controls the collection of the sensor signals, can perform additional signal processing and can compute the location parameters of the TMS coil array 200. It can also interface 220 to controller 140 to provide synchronization and other control as is known in the art. Processor 190 can also provide user interface 230 such as accepting user commands, outputting of the location parameters, etc. In the preferred embodiment, processor 190 is a digital signal processor (DSP), although any device capable of computation, such as a microprocessor, processor core, application specific integrated circuit (ASIC), gate array, personal computer, etc. may be used. The processor 190 also contains storage for data, software algorithms, etc. The preferred user interface 230 is a monitor and keyboard, but direct connection to another computer via USB or Ethernet is possible. Processor 190 interfaces to other tracker 240 components such as A/D 180, signal processor 170 over an internal bus.

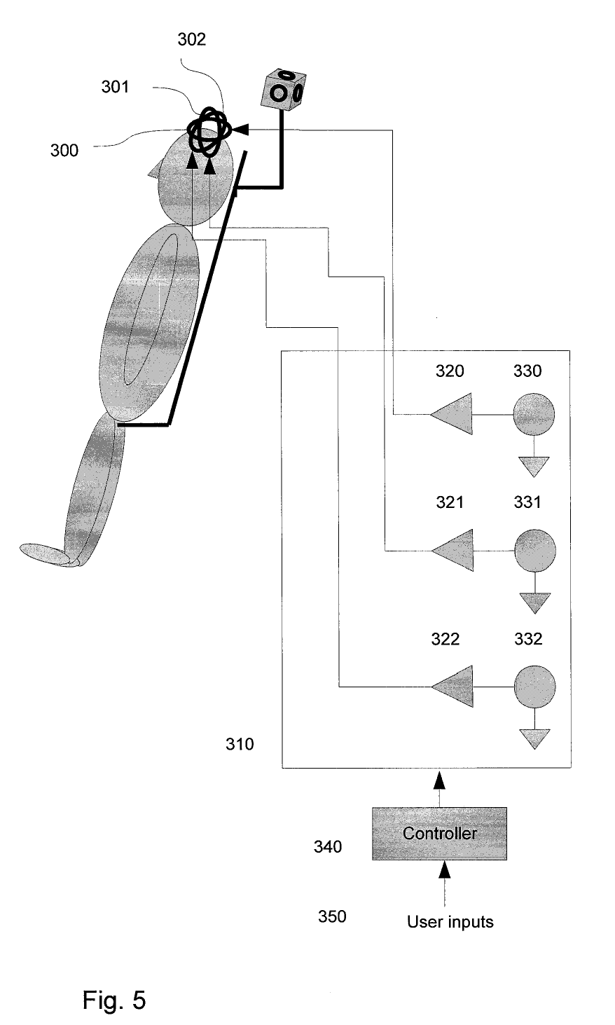

There are many TMS coil designs used to help focus the magnetic field. There are round coils, FIG. 8 coils, double cone and H coils. Coils beyond the round type are designed to better focus the magnetic field. In another preferred embodiment (FIG. 5), the TMS coil is comprised of 3, mostly orthogonal, mostly concentric coils (300, 301, 302), each with individually controlled excitations via 310. Generator 310 is comprised of waveform sources 330, 331, 332 and a means of generating high currents and voltages, symbolized by 320-322. The generators 330-332, in turn, are controlled by controller 340, which sets waveform generator amplitudes, durations, and repetition rates, and provides synchronization and other capabilities to the user. User inputs 350 set the aforementioned parameters and may be manually and/or algorithmically set. By controlling the excitation to the three coils, a well controlled field vector can be established with higher accuracy than presently possible. Because all 3 coils are on at once, this coils location parameters would be limited to 5DOF operation. Other coil arrangements and excitations are also possible. In the preferred embodiment, controller 340 adjusts the three coil excitations to achieve the required magnetic field vector, and passes the current measurements from resistor 326-328 to controller 340 via multiplexer 360 and A/D 280. Controller 340 then communicates with processor 190 via communication link 320 to provide this information.

As is known in the art, there are many calibration methods used for calibrating transmitters and sensors that comprise a magnetic tracking system. Dipole modeling refinements such as found in U.S. Pat. No. 6,335,617 to Osadchy are possible, among others and are incorporated herein. In a further embodiment, transmitter field generation can be calibrated using a mapping of the generated fields. The TMS coil(s) can be excited to produce field in their normal manner and a measurement of the magnetic field over a volume (including at least the head) could be made. This data would then be stored in some form of computer memory and an interpolation algorithm residing in a computer processor could determine the applied field at a given location from the TMS coil(s). This technique could also supplement other calibration methods, including dipole modeling, Legendre polynomials, etc. Methods of this sort are disclosed in U.S. Pat. No. 6,427,079 to Schneider, and a clone of Schneider's, U.S. Pat. No. 6,484,118, to Govari, and are incorporated herein. While these methods do not guarantee a knowledge of the location of the excitation of the neurons in the cortex, they still provide a more accurate model of the magnetic field geometry.

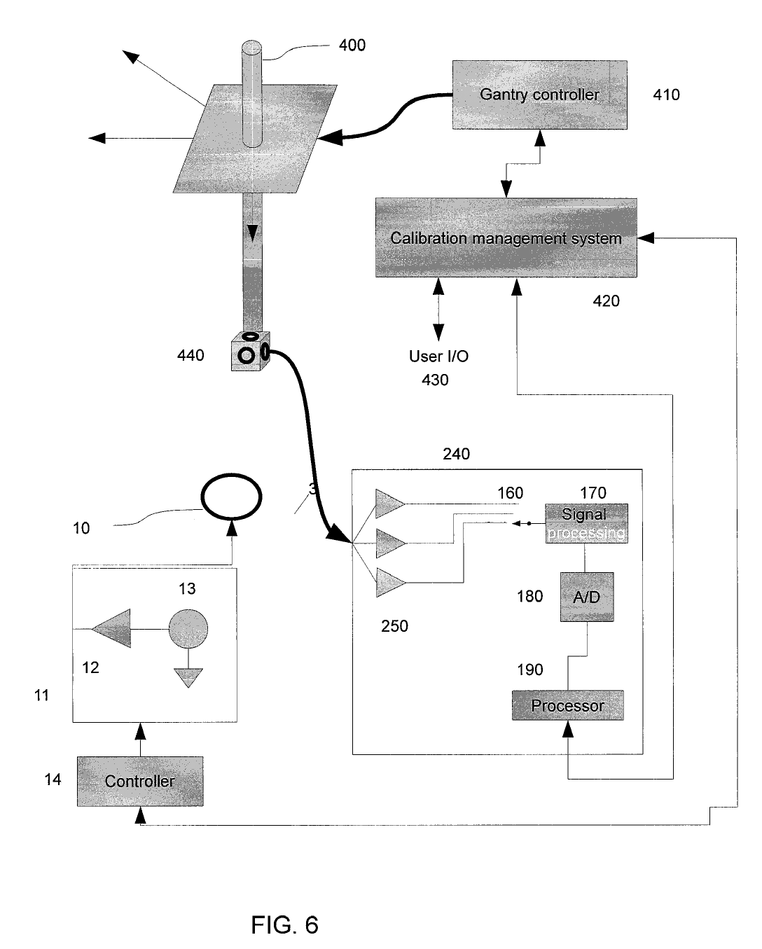

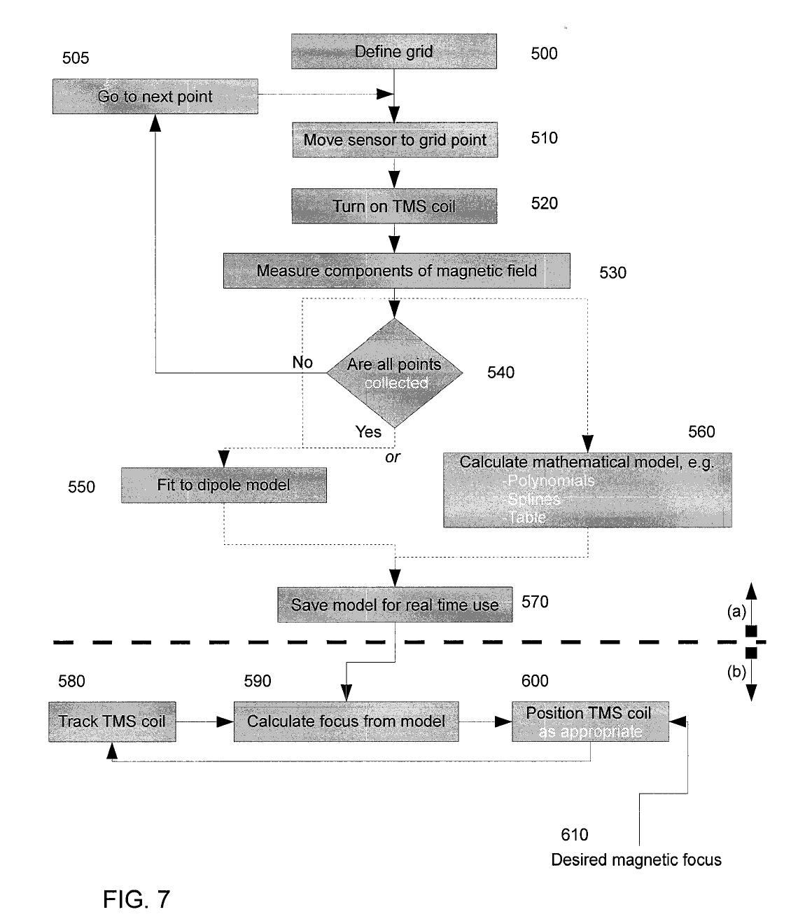

FIG. 6 shows the preferred embodiment for collecting this data. Calibration management system 420 is a computer based system that controls the entire data collection process. It commands the gantry 400 to the required 3D coordinates in Cartesian coordinate space by issuing commands to the gantry controller 410. Gantry controller 410 can be incorporated into the computer or could be a stand alone device that communicates with 420 via a standard interface 405 such as USB. This places the 3 coil sensor 440 at the required location for collecting the 3 Cartesian components of magnetic field generated by TMS coil 10, which is placed at a fixed location. The calibration management system 420 commands the TMS coil 10 to be excited via 11 (described previously), thus generating a magnetic field. Sensor 440 is typically a 3 axis sensor, with the sensing elements both concentric, collocated and orthogonal, as is known in the art. The sensed magnetic field signals are collected using electronics 240 (described previously) and this data is saved by calibration management system 420 along with the location of the sensor (as placed by the gantry) and possibly other system and setup parameters. The data collected is turned into parameters that describe the TMS magnetic field geometry, which is then used to help aim the focus of the TMS coil. These parameters can describe models or tables for interpolation, as is known in the art. A flowchart for this process is given in FIG. 7.

The FIG. 7(a) flowchart covers blocks that would normally run on a computer, are inputs or outputs to a computer, or represent a sequence of events that occur via automation. It is understood that other devices might provide the same functionality. In the preferred embodiment, user inputs 430 into the calibration management system 420 (FIG. 6) are used to define the three dimensional grid that the magnetic field data will be collected on. This is shown in block 500. Once defined, the sensor is moved to each grid point (block 510). At each grid point, the TMS coil is turned on (520) and the magnetic field components are measured (530). This would be performed by electronics blocks 11 and 14, and electronic block 240, respectively, of FIG. 6. The sensor would move to all grid points (540 and 505) until all the data was collected. Once collected, a predetermined model would be produced, such as a dipole model (550) or any number of mathematical models and/or structures (560), as are known in the art. In the preferred embodiment, this data is stored in interpolation functions known as splines. These structures are then saved (570) for each TMS coil for use during a TMS procedure. This data would be saved in a storage device that travels with the coil, but could reside elsewhere.

During a TMS procedure, FIG. 7(b), this data (570) would be made available to the TMS system, exemplified by FIG. 1. The TMS coil 10 would be tracked in 580, and knowing its location parameters, its focus can be calculated in block 590. The desired focus, from block 610, would be compared to the calculated focus (590) in block 600 and adjustments to the TMS coils physical location would be made until the desired and calculated focus are identical. The desired focus can come from other imaging modalities of the brain and/or feedback from monitoring evoked potentials or limb movement, as noted previously.

It is appreciated that various features of the invention which are, for clarity, described in the contexts of separate embodiments may also be provided in combination in a single embodiment. Conversely, various features of the invention which are, for brevity, described in the context of a single embodiment may also be provided separately on or in any suitable sub-combination.

It will be appreciated by persons skilled in the art that the present invention is not limited to what has been particularly shown and described herein. Rather, the scope of the present invention is defined only by the claims that follow.

What is claimed is:

1. A system for tracking the location of a magnetic stimulation coil, the system comprising:
a magnetic stimulation single coil system operative to provide a health benefit; and
a plurality of sensing devices, fixed in a known location, operative to detect the magnetic field from said magnetic stimulation single coil system; and
a processing means for assembling the detected magnetic field data into location parameters of the stimulation coil with respect to the sensing devices

2. A system according to claim 1 wherein the sensors are coplanar

3. A system according to claim 1 wherein the sensors are not coplanar

4. A system according to claim 3 wherein the sensors are orthogonal

5. A system according to claim 3 wherein the sensors are co-located

6. A system according to claim 1 wherein the sensors are both coplanar and not coplanar

7. A system for tracking the location of a magnetic stimulation coil, the system comprising:
a magnetic stimulation single coil system operative to provide a health benefit; and
a plurality of sensing devices, fixed in a known location, operative to detect the magnetic field from said magnetic stimulation single coil system; and
a plurality of sensing devices, mounted to a mobile device, operative to detect the magnetic field from said magnetic stimulation single coil system; and
a processing means for assembling the detected magnetic field data into location parameters of the stimulation coil with respect to the sensing devices

8. A system according to claim 7 wherein the sensors are coplanar

9. A system according to claim 7 wherein the sensors are not coplanar

10. A system according to claim 9 wherein the sensors are orthogonal

11. A system according to claim 9 wherein the sensors are co-located

12. A system according to claim 7 wherein the sensors are both coplanar and not coplanar

13. A system according to claim 7 wherein the sensors are attached to a stylus

14. A system according to claim 7 wherein the sensors are attached to a physiological detector.

15. A system for tracking the location of a magnetic stimulation coil, the system comprising:
a magnetic stimulation single coil system operative to provide a health benefit; and
a plurality of field generating coils located in a known fixed manner to the magnetic stimulation coil; and
a plurality of sensing devices, fixed in a known location, operative to detect the magnetic field from said magnetic stimulation single coil system; and
a processing means for assembling the detected magnetic field data into location parameters of the stimulation coil with respect to the sensing devices

16. A system according to claim 15 wherein the additional field generating coils are coplanar

17. A system according to claim 15 wherein the additional field generating coils are not coplanar

18. A system according to claim 17 wherein the additional field generating coils are orthogonal

19. A system according to claim 17 wherein the additional field generating coils are co-located

20. A system according to claim 15 wherein the additional field generating coils are both coplanar and not coplanar

21. A system according to claim 15 wherein the sensors are coplanar

22. A system according to claim 15 wherein the sensors are not coplanar

23. A system according to claim 22 wherein the sensors are orthogonal

24. A system according to claim 22 wherein the sensors are co-located

25. A system according to claim 15 wherein the sensors are both coplanar and not coplanar

26. A system for tracking the location of a set of magnetic stimulation coil, the system comprising:
a magnetic stimulation system operative to provide a health benefit; and
a plurality field generating coils located in a known fixed manner to the magnetic stimulation coil; and
a plurality of sensing devices, fixed in a known location, operative to detect the magnetic field from said magnetic stimulation single coil system; and
a plurality of sensing devices, mounted to a mobile device, operative to detect the magnetic field from said magnetic stimulation single coil system; and
a processing means for assembling the detected magnetic field data into location parameters of the stimulation coil with respect to the sensing devices

27. A system according to claim 26 wherein the additional field generating coils are coplanar

28. A system according to claim 26 wherein the additional field generating coils are not coplanar

29. A system according to claim 28 wherein the additional field generating coils are orthogonal

30. A system according to claim 28 wherein the additional field generating coils are co-located

31. A system according to claim 26 wherein the additional field generating coils are both coplanar and not coplanar

32. A system according to claim 26 wherein the sensors are coplanar

33. A system according to claim 26 wherein the sensors are not coplanar

34. A system according to claim 33 wherein the sensors are orthogonal

35. A system according to claim 33 wherein the sensors are co-located

36. A system according to claim 26 wherein the sensors are both coplanar and not coplanar

37. A system according to claim 26 wherein the sensors are attached to a stylus

38. A system according to claim 26 wherein the sensors are attached to a physiological detector.

39. A system according to claim 26 wherein the additional field generating coils provide a magnetic stimulation in a more focused manner

40. A system for calibrating the focus of a magnetic stimulation coil, the system comprising:
a magnetic stimulation system operative to provide a health benefit; and
a plurality of sensing devices, movable to known locations, operative to detect the magnetic field from said magnetic stimulation system; and
a processing means for assembling the detected magnetic field data into a mathematical construct; and a means for storing the construct for in a magnetic stimulation system

41. A system according to claim 40 wherein the construct is accessed to yield magnetic field focus information for the purpose of placing the stimulation coils in an optimum location
